(19) United States
(12) Patent Application Publication
(10) Pub. No.: US 2019/0275199 A1
(43) Pub. Date: Sep. 12, 2019

(54) PRINTING OF BIOPOLYMERS FROM IONIC LIQUID

(71) Applicants: THE BOARD OF TRUSTEES OF THE UNIVERSITY OF ALABAMA, Tuscaloosa, AL (US); 525 Solutions, Inc., Tuscaloosa, AL (US)

(72) Inventors: Robin D. Rogers, Tuscaloosa, AL (US); Oleksandra Zavgorodnya, Tuscaloosa, AL (US); Julia L. Shamshina, Tuscaloosa, AL (US); Gabriela Gurau, Tuscaloosa, AL (US)

(21) Appl. No.: 16/296,776

(22) Filed: Mar. 8, 2019

Related U.S. Application Data
(60) Provisional application No. 62/641,038, filed on Mar. 9, 2018.

(51) Int. Cl. A61L 27/18; A61L 27/20; B29C 64/118; B33Y 10/00; B33Y 70/00

(52) U.S. Cl. CPC A61L 27/18; A61L 27/20; B29K 2067/046; B33Y 10/00; B33Y 70/00; B29C 64/118

(57) ABSTRACT

Compositions and methods of printing a three-dimensional (3D) article from a printing composition comprising a biopolymer are described. In addition to the biopolymer, the printing composition includes an ionic liquid solvent and optionally, a synthetic polymer. The method of printing the 3D article includes extruding the printing composition from a deposition nozzle moving relative to a substrate, depositing one or more layers comprising the printing composition in a predetermined pattern on the substrate, and treating the one or more layers to form the 3D article. The one or more layers deposited on the substrate can exhibit sufficient stiffness to maintain its shape once deposited, thus depositing the printing composition into a mold is not required. Treating the one or more layers can comprise coagulating the biopolymer and/or removing the ionic liquid solvent using an aqueous solvent.

### CROSS-REFERENCE TO RELATED APPLICATIONS

This application claims priority to U.S. Provisional Patent Application No. 62/641,038, filed Mar. 9, 2018, and entitled “PRINTING OF BIOPOLYMERS FROM IONIC LIQUID,” the entire disclosure of which is incorporated herein by reference.

### FIELD

The present disclosure generally relates to printing biopolymers, particularly to printing biopolymers from ionic liquids.

### BACKGROUND

The accumulation of non-degradable polymeric waste in both landfills and oceans, as well as the finite amount of petroleum resources are major environmental and economic concerns raised in the last decade (Thompson, R. C. et al., Phil. Trans. R. Soc. B 2009, 364, 2153-2166; Brehmer, B. et al., Chem. Eng. Res. Des. 2009, 87, 1103-1119). Indeed, since 1950 more than 7 billion tons of plastics have been produced, and as of today, there are more than 250,000 tons of it floating in the world oceans (Eriksen, M. et al., PLoS ONE 2014, 9, e111913). If plastic production remains at this rate, the ocean will contain more plastic than fish by 2050.

Biomass feedstocks (such as wood and animal residues) have been shown to be a useful substitute for synthetic plastics and provide a more sustainable approach to make different products (Nagahama, H. et al., Carbohydr. Polym. 2008, 73, 295-302; Pillai, C. K. S. et al., Prog. Polym. Sci. 2009, 34, 641-678; Tamura, H. et al., Carbohydr. Polym. 2011, 84, 820-824; Flores, R. et al., J. Appl. Polym. Sci. 2007, 104, 3909-3916). Among the different polymers found in biomass, chitin and cellulose are two of the most abundant ones that provide biodegradability and low toxicity. In particular, chitin offers the possibility of surface functionalization, good antibacterial activity, wound healing, and bone regenerating properties (Vázquez, J. A. et al., Mar. Drugs 2013, 11, 747-774; Mori, T. et al., Biomaterials, 1997, 18, 947-951; Jayakumar, R. et al., In Biomedical Engineering, Trends in Materials Science, ed. A. N. Laskovski, Intech, 2011, Chapter 1, pp. 3-24). This makes chitin attractive as a material for use in tissue engineering (Lee, K. Y. et al., Adv. Drug Deliv. Rev. 2009, 1020-1032; Singh, N. et al., Nanoscale 2016, 8, 8288-8299), drug delivery (Mi, F. L. et al., Biomaterials 2003, 24, 5023-5036), and for metal recovery (Barber, P. S. et al., Green Chem. 2014, 16, 1828-1836; Schleuter, D. et al., Carbohydr. Polym. 2013, 92, 712-718). Similarly, cellulose is known for its remarkable mechanical properties and is currently used in industrial applications to reinforce synthetic plastics (Eichhorn, S. J. et al., Cellulose 2001, 8, 197-207).

Biopolymers extracted from natural sources are usually insoluble in conventional solvents due to their high degree of crystallinity. Therefore, biopolymers generally cannot be melt-processed into advanced materials using traditional processing methods generally used for thermoplastics. Moreover, biopolymers manufactured into advanced materials through the utilization of harsh chemicals to degrade the biopolymer are generally unsuitable for manufacturing biomedical materials (Muzzarelli, R. A. A. Carbohydr. Polym. 1983, 3, 53-75; Seoudi, R. et al., Carbohydr. Polym. 2007, 68, 728-33). Alternative approaches for processing biopolymers into advanced materials suitable for medical applications are needed. The compositions and methods disclosed herein address these and other needs.

### SUMMARY

In accordance with the purposes of the disclosed compositions and methods, as embodied and broadly described herein, the disclosed subject matter relates to compositions and methods of printing a three-dimensional (3D) article from a biopolymer. The printing composition can comprise the biopolymer dissolved in an ionic liquid solvent. The method can include extruding the printing composition from a deposition nozzle moving relative to a substrate, depositing one or more layers comprising the printing composition in a predetermined pattern on the substrate, and treating the one or more layers to form the 3D article. Extruding the printing composition can be carried out at ambient temperature or greater, preferably from 20° C. to 150° C., more preferably from ambient temperature to 60° C., or from 30° C. to 50° C.

The biopolymer used in the printing compositions can include starch, pectin, chitin, chitosan, alginate, silk, elastin, collagen, gelatin, hemicellulose, lignin, cellulose, lignocellulose, or combinations thereof. In some examples, the biopolymer includes a regenerated biomass, such as regenerated chitin. The biopolymer can be present in the printing composition in an amount from 0.1 wt % to 50 wt %, from 0.1 wt % to 25 wt %, or from 1 wt % to 15 wt %.

The ionic liquid can comprise a cation and an anion, wherein the cation is selected from the group consisting of:

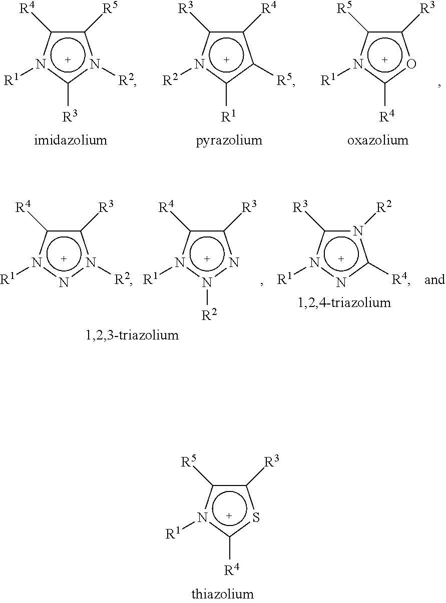

wherein each R¹ and R² is, independently, a substituted or unsubstituted linear, branched, or cyclic C₁-C₆ alkyl, or substituted or unsubstituted linear, branched, or cyclic C₁-C₆ alkoxy;
each R³, R⁴, and R⁵ is, independently, hydrogen, substituted or unsubstituted linear, branched, or cyclic C₁-C₆ alkyl, substituted or unsubstituted linear, branched, or cyclic C₁-C₆ alkoxy, or substituted or unsubstituted linear or branched, C₁-C₆ alkoxyalkyl; and
wherein the anion is selected from the group consisting of C₁₋₆ carboxylate, halide, CO₃²; NO₂⁻, NO₃⁻, SO₄²⁻, CN⁻, R¹⁰CO₂, (R¹⁰O)₂P(═O)O, (R¹⁰O)S(═O)₂O, or (R¹⁰O)C(═O)O; where R¹⁰ is hydrogen; substituted or unsubstituted linear, branched, or cyclic alkyl;

substituted or unsubstituted linear, branched, or cyclic alkoxy; substituted or unsubstituted aryl; substituted or unsubstituted aryloxy; substituted or unsubstituted heterocyclic; and substituted or unsubstituted heteroaryl.

In certain embodiments, the ionic liquid comprises an imidazolium cation, such as a 1-alkyl-3-alkyl imidazolium C₁-C₆ carboxylate. In some examples, the ionic liquid includes 1-ethyl-3-methyl-imidazolium acetate ([C₂mim]OAc).

The printing composition can further include a co-solvent. In some embodiments, the printing composition does not include a co-solvent, such as an organic solvent selected from 1-butanol or dimethyl sulfoxide (DMSO).

The printing composition can further comprise a synthetic polymer. The synthetic polymer can be selected from a polylactic acid, a polyester, a polyacrylonitrile, a poly(N,N-dimethyl acrylamide), a poly(l-vinylpyrrolidinone), a polyhydroxyethylmethacrylate, a polymethylmethacrylate, a poly(vinylidene fluoride), a polycaprolactone, a polyalkylene glycol, a polyurethane, or a combination thereof. The biopolymer and the synthetic polymer can be in a weight ratio of from 1:0.1 to 1:20, preferably from 1:1 to 1:20, more preferably from 1:1 to 1:10.

The printing composition can further comprise biologically active compounds, plasticizers, pigments, fire retardants, catalysts, cross-linkers, heat or light stabilizers, organic or inorganic fillers such as nano-fillers, fiber reinforcements, or combinations thereof. In certain embodiments, the printing composition comprises a nano-filler selected from carbon nanotubes, graphene nanoplatelets and flakes, graphite powder, clay, metals and nanoparticles agents or dopants, or a combination thereof.

In printing the 3D article, the deposition nozzle can move relative to the substrate at a print speed from about 1 mm/s to about 100 mm/s, preferably from about 10 mm/s to about 50 mm/s. The one or more layers deposited on the substrate can exhibit sufficient stiffness to maintain its shape once deposited. Accordingly, the methods described herein does not require depositing the printing composition into a mold.

Prior to treating the one or more layers, the methods disclosed herein can include allowing the one or more layers to solidify at 35° C. or less, such as ambient temperature or less, 25° C. or less, or from 0° C. to 25° C. Treating the one or more layers can comprise coagulating the biopolymer and/or removing the ionic liquid solvent. In some embodiments, coagulating the biopolymer and/or removing the ionic liquid solvent can include contacting the one or more layers with a non-solvent. The non-solvent can include an aqueous solvent such as water. Coagulating the biopolymer can occur after printing when the 3D article is near dry. Three-dimensional (3D) printed article derived from the compositions and methods described herein are also disclosed. The articles can be used in optoelectronics, photonics, therapeutics, tissue engineering such as intelligent implants, or synthetic biology.

Printing compositions consisting essentially of a biopolymer in an amount of from 0.1 wt % to 50 wt %, based on the weight of the printing composition, a synthetic polymer, wherein the biopolymer and the synthetic polymer are in a weight ratio of from 1:0.1 to 1:20, preferably from 1:1 to 1:20, more preferably from 1:1 to 1:10, an ionic liquid solvent for dissolving the biopolymer and synthetic polymer, and a 3D printing additive such as biologically active compounds, plasticizers, pigments, fire retardants, catalysts, cross-linkers, heat or light stabilizers, organic or inorganic fillers such as nano-fillers, fiber reinforcements, or combinations thereof are also disclosed herein.

Additional advantages of the disclosed process will be set forth in part in the description which follows, and in part will be obvious from the description, or can be learned by practice of the disclosed process. The advantages of the disclosed process will be realized and attained by means of the elements and combinations particularly pointed out in the appended claims. It is to be understood that both the foregoing general description and the following detailed description are exemplary and explanatory only and are not restrictive of the disclosed process, as claimed.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The accompanying figures, which are incorporated in and constitute a part of this specification illustrate several aspects described below.

FIGS. 1A-1B are photographs showing three-dimensional (3D) printed chitin material (40 cm diameter) on a solid support prior to (FIG. 1A) and after (FIG. 1B) coagulating in an anti-solvent.

FIGS. 2A-2B are photographs showing three-dimensional printed chitin ring after coagulating for 30 min in an aqueous bath (FIG. 2A) and after complete release of ionic liquid (FIG. 2B).

FIG. 3 is a photograph showing a cubical modeled three-dimensional printed chitin material after coagulation and ionic liquid removal.

FIGS. 4A-4B are photographs showing three-dimensional printed chitin material after freeze-drying.

FIGS. 5A-5C are photographs showing three-dimensional printed chitin-polylactic acid (PLA) material (chitin to PLA ratio is 1:1) prior to (FIG. 5A), during (FIG. 5B), and after (FIG. 5C) coagulation.

### DETAILED DESCRIPTION

The materials, compounds, compositions, articles, and methods described herein can be understood more readily by reference to the following detailed description of specific aspects of the disclosed subject matter and the Examples and Figures included therein.

Before the present materials, compounds, compositions, articles, devices, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an ionic liquid” includes mixtures of two or more such ionic liquids, reference to “the biopolymer” includes mixtures of two or more such biopolymers, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed, then “less than or equal to” the value, “greater than or equal to the value,” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

### Chemical Definitions

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt %) of a component, unless specifically stated to the contrary, is based on the total weight of the formulation or composition in which the component is included.

The term “ion,” as used herein, refers to any molecule, portion of a molecule, cluster of molecules, molecular complex, moiety, or atom that contains a charge (positive, negative, or both at the same time within one molecule, cluster of molecules, molecular complex, or moiety (e.g., zwitterions)) or that can be made to contain a charge. Methods for producing a charge in a molecule, portion of a molecule, cluster of molecules, molecular complex, moiety, or atom are disclosed herein and can be accomplished by methods known in the art, e.g., protonation, deprotonation, oxidation, reduction, alkylation acetylation, esterification, deesterification, hydrolysis, etc.

The term “anion” is a type of ion and is included within the meaning of the term “ion.” An “anion” is any molecule, portion of a molecule (e.g., zwitterion), cluster of molecules, molecular complex, moiety, or atom that contains a net negative charge or that can be made to contain a net negative charge.

The term “cation” is a type of ion and is included within the meaning of the term “ion.” A “cation” is any molecule, portion of a molecule (e.g., zwitterion), cluster of molecules, molecular complex, moiety, or atom, that contains a net positive charge or that can be made to contain a net positive charge.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, 1-ethyl-2-methyl-propyl, heptyl, octyl, nonyl, decyl, dodecyl, tetradecyl, hexadecyl, eicosyl, tetracosyl, and the like. Alkyl substituents may be unsubstituted or substituted with one or more chemical moieties. The alkyl group can be substituted with one or more groups including, but not limited to, hydroxyl, halogen, acyl, alkyl, alkoxy, alkenyl, alkynyl, aryl, heteroaryl, aldehyde, amino, cyano, carboxylic acid, ester, ether, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “aryl,” as well as derivative terms such as aryloxy, refers to groups that include a monovalent aromatic carbocyclic group of from 3 to 50 carbon atoms. Aryl groups can include a single ring or multiple condensed rings. In some embodiments, aryl groups include C₆-C₁₀ aryl groups. Examples of aryl groups include, but are not limited to, benzene, phenyl, biphenyl, naphthyl, tetrahydronaphtyl, phenylcyclopropyl, phenoxybenzene, and indanyl. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is also included in the term “aryl,” defines a group that contains an aromatic group that does not contain a heteroatom. The aryl substituents may be unsubstituted or substituted with one or more chemical moieties. Examples of suitable substituents include, for example, alkyl, alkoxy, alkenyl, alkynyl, aryl, heteroaryl, acyl, aldehyde, amino, cyano, carboxylic acid, ester, ether, halide, hydroxyl, ketone, nitro, phosphonyl, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

As used herein, the term “alkoxy” as used herein is an alkyl group bound through a single, terminal ether linkage; that is, an “alkoxy” group can be defined as to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “halide” or “halogen” or “halo” as used herein refers to fluoro, chloro, bromo, and iodo radicals.

The term “hydroxyl” as used herein is represented by the formula —OH.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

The term “hydrogen bond” describes an attractive interaction between a hydrogen atom from a molecule or molecular fragment X—H in which X is more electronegative than H, and an atom or a group of atoms in the same or different molecule, in which there is evidence of bond formation. The hydrogen bond donor can be a cation and the hydrogen bond acceptor can be an anion.

The term “complex” describes a coordination complex, which is a structure comprised of a central atom or molecule that is weakly connected to one or more surrounding atoms or molecules, or describes chelate complex, which is a coordination complex with more than one bond.

References to “mim,” “Cₙ-mim,” and “bmim” are intended to refer to a methyl imidazolium compound, an alkyl (with n carbon atoms) methyl imidazolium compound, and a butyl methylimidazolium compound respectively.

As used herein, the term “biopolymer biomass” refers to any source of biopolymer (such as cellulose, chitin or chitosan) that is derived from a natural resource (e.g., wood, animal residue, or microorganism).

Reference will now be made in detail to specific aspects of the disclosed materials, compounds, compositions, formulations, articles, and methods, examples of which are illustrated in the accompanying Examples and Figures.

Compositions

Disclosed herein are compositions and methods for printing three-dimensional (3D) articles. Particularly, printing compositions comprising a biopolymer solubilized in an ionic liquid are described herein. Methods of making and using the printing compositions are also described.

Biopolymers

The biopolymer for use in the compositions described herein can be any biopolymer either in a processed, derivatized, pure, or unpure form. Non-limiting examples of biopolymers include starch, pectin, chitin, chitosan, alginate, silk, elastin, collagen, gelatin, hemicellulose, lignin, cellulose, or a mixture thereof. In some examples, the biopolymer can be lignin and hemicelluloses bonded or unbonded lignocellulosic biomass, such as hemp. In a preferred aspect, the biopolymer is chitin.

In certain embodiments, the biopolymer can be present in a biomass and the biomass can be mixed directly with the ionic liquid mixtures. Thus, disclosed are compositions comprising biomass and an ionic liquid. Also described are methods for dissolving biomass in the ionic liquid mixtures. In this aspect, the biomass used can be fractioned, treated, derivatized, and/or otherwise processed. The term “biomass,” as used herein, refers to living or dead biological material that can be used in one or more of the disclosed processes. Biomass can comprise any of the biopolymers such as cellulosic or lignocellulosic biopolymers described herein, and optionally further comprises oligosaccharides and/or monosaccharides, other biopolymers, natural derivatives of biopolymers, their mixtures, and breakdown products (e.g., metabolites). Biomass can also comprise additional components, such as protein and/or lipid. Biomass can be derived from a single source, or biomass can comprise a mixture derived from more than one source. Some specific examples of biomass include, but are not limited to, bioenergy crops, agricultural residues, municipal solid waste, industrial solid waste, sludge from paper manufacture, yard waste, wood and forestry waste. Additional examples of biomass include, but are not limited to, corn grain, corn cobs, crop residues such as corn husks, corn stover, grasses, wheat, wheat straw, hay, rice straw, switchgrass, waste paper, sugar cane bagasse, sorghum, soy, components obtained from milling of grains, trees (e.g., pine), branches, roots, leaves, wood chips, wood pulp, sawdust, shrubs and bushes, vegetables, fruits, flowers, animal manure, multi-component feed, and crustacean biomass (e.g., chitinous biomass from shellfish, shrimp and/or crab shells).

Lignocellulosic biomass typically comprises of three major components: cellulose, hemicellulose, and lignin, along with some extractive materials (Sjostorm, E. Wood Chemistry: Fundamentals and Applications, 2nd ed., 1993, New York.). Depending on the source, their relative compositions usually vary to certain extent. Cellulose is the most abundant polymer on Earth and enormous effort has been put into understanding its structure, biosynthesis, function, and degradation (Stick, R. V. Carbohydrates—The Sweet Molecules of Life, 2001, Academic Press, New York.). Cellulose is actually a polysaccharide consisting of linear chain of several hundred to over ten thousand β(1→4) linked D-glucose units. The chains are hydrogen bonded either in parallel or anti-parallel manner which imparts more rigidity to the structure, and a subsequent packaging of bound-chains into microfibrils forms the ultimate building material of the nature.

Hemicellulose is the principal non-cellulosic polysaccharide in lignocellulosic biomass. Hemicellulose is a branched heteropolymer, consisting of different sugar monomers with 500-3000 units. Hemicellulose is usually amorphous and has higher reactivity than the glucose residue because of different ring structures and ring configurations. Lignin is the most complex naturally occurring high-molecular weight polymer (Hon, D. N. S.; Shiraishi, N., Eds., Wood and Cellulosic Chemistry, 2^{nd} ed., 2001, Marcel Dekker, Inc., New York.). Lignin is relatively hydrophobic and aromatic in nature, but lacks a defined primary structure. Softwood lignin primarily comprises guaiacyl units, and hardwood lignin comprises both guaiacyl and syringyl units. Cellulose content in both hardwood and softwood is about 43±2%. Typical hemicellulose content in wood is about 28-35 wt %, depending on type of wood. Lignin content in hardwood is about 18-25% while softwood may contain about 25-35% of lignin.

Chitin is a polymer of N-acetyl-D-glucosamine and has a similar structure to cellulose. It is an abundant polysaccharide in nature, comprising the horny substance in the exoskeletons of crab, shrimp, lobster, cuttlefish, and insects as well as fungi. Any of these or other sources of chitin are suitable for use in the methods and compositions disclosed herein. In addition to chitin, chitin derivatives can be used. One such derivative is chitosan. Chitosan is a de-acetylated form of chitin and occurs naturally in some fungi.

Synthetic Polymers

The compositions described herein can further include a synthetic polymer. In one aspect, the synthetic polymer can comprise hydrogen bond donors and/or hydrogen bond acceptors. Examples of such polymers include those comprising hydroxyl, amino, amido, carbonyl, or ester functional groups, for example. The synthetic polymer can be derived from polymer materials including polylactic acid (PLA), a polycaprolactone, a polyester, a polyacrylonitrile, a poly(N,N-dimethyl acrylamide), a poly(l-vinylpyrrolidinone), a polyhydroxyethylmethacrylate, a polymethylmethacrylate, a poly(vinylidene fluoride), a polycaprolactone, a polyalkylene glycol such as polyethylene glycol and polypropylene glycol, a polyalkyleneamine such as polyethyleneamine, a polyurethane, a polyamide, a polyimideamide, a polybenzoimide, an aramide, a polyimide, or a combination thereof.

In some cases, the synthetic polymer can include a biodegradable synthetic polymer. In some aspects, the disclosed compositions comprise the synthetic polymer in an amount of 0.5 wt % or greater (e.g., 0.75 wt % or greater, 1 wt % or greater, 1.5 wt % or greater, 2 wt % or greater, 2.5 wt % or greater, 3 wt % or greater, 3.5 wt % or greater, 4 wt % or greater, 4.5 wt % or greater, 5 wt % or greater, 5.5 wt % or greater, 6 wt % or greater, 7 wt % or greater, 8 wt % or greater, 9 wt % or greater, 10 wt % or greater, 15 wt % or greater, 20 wt % or greater, 25 wt % or greater, 30 wt % or greater, 35 wt % or greater, or 40 wt % or greater), based on the total weight of the composition. In some aspects, the disclosed compositions comprise the synthetic polymer in an amount of 75 wt % or less (e.g., 60 wt % or less, 65 wt % or less, 60 wt % or less, 55 wt % or less, 50 wt % or less, 45 wt % or less, 40 wt % or less, 30 wt % or less, 25 wt % or less, 20 wt % or less, 15 wt % or less, 10 wt % or less, 7.5 wt % or less, 5 wt % or less, 4.5 wt % or less, 4 wt % or less, 3.5 wt % or less, 3 wt % or less, or 2.5 wt % or less), based on the total weight of the composition. In some aspects, the disclosed compositions comprise the synthetic polymer in an amount of from 0.5 wt % to 75 wt %, from 0.5 wt % to 50 wt %, from 0.5 wt % to 25 wt %, from 0.5 wt % to 20 wt %, from 0.5 wt % to 10 wt %, from 1 wt % to 10 wt %, or from 1 wt % to 5 wt %), based on the total weight of the composition.

Ionic Liquids

As described herein, the compositions include the biopolymer and optional synthetic polymer solubilized in an ionic liquid solvent. The term “ionic liquid” has many definitions in the art, but is used herein to refer to salts (i.e., an ionic compound of cations and anions) that are liquid at a temperature of at or below about 150° C. That is, at one or more temperature ranges or points at or below about 150° C. the disclosed ionic liquid compositions are liquid; although, it is understood that they can be solids at other temperature ranges or points. See e.g., Wasserscheid and Keim, Angew Chem Int Ed Engl, 2000, 39:3772; and Wasserscheid, “Ionic Liquids in Synthesis,” 1^{st} Ed., Wiley-VCH, 2002.

Ionic liquids can possess an extremely strong hydrogen bond basicity necessary to disrupt the hydrogen bonding network of natural biopolymers like those mentioned herein. In addition to the effective dissolution and easy regeneration of biopolymers by precipitation, upon addition of water or other common solvents, ionic liquids also prevent their degradation.

In some examples, the ionic liquid can be a liquid at a temperature of about 150° C. or less (e.g., about 140° C. or less, about 130° C. or less, about 120° C. or less, about 110° C. or less, about 100° C. or less, about 90° C. or less, about 80° C. or less, about 70° C. or less, about 60° C. or less, about 50° C. or less, about 40° C. or less, about 30° C. or less, about 20° C. or less, about 10° C. or less, about 0° C. or less, about −10° C. or less, about −20° C. or less, or about −30° C. or less). Further, in some examples the disclosed ionic liquids can be liquid over a range of temperatures. For example, the disclosed ionic liquids can be liquids over a range of about 1° C. or more (e.g., about 2° C. or more, about 3° C. or more, about 4° C. or more, about 5° C. or more, about 6° C. or more, about 7° C. or more, about 8° C. or more, about 9° C. or more, about 10° C. or more, about 11° C. or more, about 12° C. or more, about 13° C. or more, about 14° C. or more, about 15° C. or more, about 16° C. or more, about 17° C. or more, about 18° C. or more, about 19° C. or more, or about 20° C. or more). Such temperature ranges can begin and/or end at any of the temperature points disclosed above.

In further examples, the disclosed ionic liquids can be liquid at temperature from about −30° C. to about 150° C. (e.g., from about −20° C. to about 140° C., about −10° C. to about 130° C., from about 0° C. to about 120° C., from about 10° C. to about 110° C., from about 20° C. to about 100° C., from about 30° C. to about 90° C., from about 40° C. to about 80° C., from about 50° C. to about 70° C., from about −30° C. to about 50° C., from about −30° C. to about 90° C., from about −30° C. to about 110° C., from about −30° C. to about 130° C., from about −30° C. to about 150° C., from about 30° C. to about 90° C., from about 30° C. to about 110° C., from about 30° C. to about 130° C., from about 30° C. to about 150° C., from about 0° C. to about 100° C., from about 0° C. to about 70° C., or from about 0° to about 50° C.).

Further, exemplary properties of ionic liquids are high ionic range, non-volatility, non-flammability, high thermal stability, wide temperature for liquid phase, highly solvability, and non-coordinating. For a review of ionic liquids see, for example, Welton, Chem Rev., 99:2071-2083, 1999; and Carlin et al., Advances in Nonaqueous Chemistry, Mamantov et al. Eds., VCH Publishing, New York, 1994. These references are incorporated by reference herein in their entireties for their teachings of ionic liquids.

The term “liquid” describes the compositions that are generally in amorphous, non-crystalline, or semi-crystalline state. For example, while some structured association and packing of cations and anions can occur at the atomic level, an ionic liquid composition can have minor amounts of such ordered structures and are therefore not crystalline solids. The compositions can be fluid and free-flowing liquids or amorphous solids such as glasses or waxes at temperatures at or below 150° C.

The ionic liquids of the present disclosure can comprise an organic cation and an organic or inorganic anion. The organic cation is typically formed by alkylation of a neutral organic species capable of holding a positive charge when a suitable anion is present.

Further, the ionic liquid can be composed of at least two different ions, each of which can independently and simultaneously introduce a specific characteristic to the composition not easily obtainable with traditional dissolution and formulation techniques. Thus, by providing different ions and ion combinations, one can change the characteristics or properties of the disclosed compositions in a way not seen by simply preparing various crystalline salt forms. Examples of characteristics that can be controlled in the disclosed compositions include, but are not limited to, melting, solubility control, rate of dissolution, and a biological activity or function. It is this multi-nature/functionality of the disclosed ionic liquid compositions which allows one to fine-tune or design in very specific desired material properties. For example, the ionic liquids of the present disclosure can comprise at least one cation and at least one anion.

The organic cation of the ionic liquids disclosed herein can comprise a linear, branched, or cyclic heteroalkyl unit. The term “heteroalkyl” refers to a cation as disclosed herein comprising one or more heteroatoms chosen from nitrogen, oxygen, sulfur, boron, or phosphorous capable of forming a cation. The heteroatom can be a part of a ring formed with one or more other heteroatoms, for example, pyridinyl, imidazolinyl rings, that can have substituted or unsubstituted linear or branched alkyl units attached thereto. In addition, the cation can be a single heteroatom wherein a sufficient number of substituted or unsubstituted linear or branched alkyl units are attached to the heteroatom such that a cation is formed. For example, the cation [Cₙmim] where n is an integer of from 1 to 8 can be used. Preferably, ionic liquids with the cation [C₁₋₄mim] can be used. A particularly useful ionic liquid is 1-ethyl-3-methyl-1H-imidazol-3-ium acetate, [C₂mim]OAc, having the formulae:

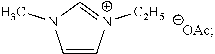

is an example of an ionic liquid comprising a cyclic heteroalkyl cation; a ring comprising 3 carbon atoms and 2 nitrogen atoms.

Other non-limiting examples of heterocyclic and heteroaryl units that can be alkylated to form cationic units include imidazole, pyrazoles, thiazoles, isothiazoles, azathiozoles, oxothiazoles, oxazines, oxazolines, oxazaboroles, dithiozoles, triazoles, selenozoles, oxahospholes, pyrroles, boroles, furans, thiphenes, phospholes, pentazoles, indoles, indolines, oxazoles, isothirazoles, tetrazoles, benzofurans, dibenzofurans, benzothiophenes, dibenzothoiphenes, thiadiazoles, pyrdines, pyrimidines, pyrazines, pyridazines, piperazines, piperidines, morpholines, pyrans, annolines, phthalazines, quinazolines, and quinoxalines.

The following are examples of heterocyclic units that are suitable for forming a cyclic heteroalkyl cation unit of the disclosed ionic liquids:

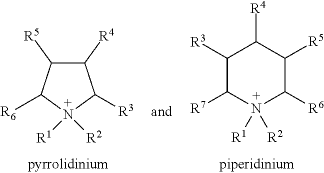

The following are further examples of heterocyclic units that are suitable for forming a cyclic heteroalkyl cation unit of the disclosed ionic liquids:

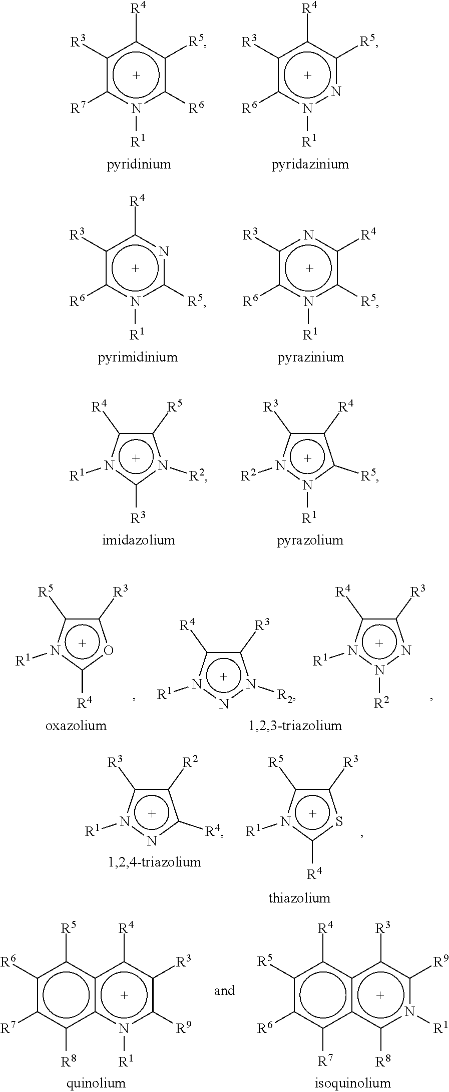

where each R¹ and R² is, independently, a substituted or unsubstituted linear, branched, or cyclic C₁-C₆ alkyl, or substituted or unsubstituted linear, branched, or cyclic C₁-C₆ alkoxy; each R³, R⁴, R⁵, R⁶, R⁷, R⁸, and R⁹ is, independently, hydrogen, substituted or unsubstituted linear, branched, or cyclic C₁-C₆ alkyl, substituted or unsubstituted linear, branched, or cyclic C₁-C₆ alkoxy, or substituted or unsubstituted linear or branched, C₁-C₆ alkoxyalkyl.

The following comprises yet another set of examples of heterocyclic units that are suitable for forming heterocyclic dication units of the disclosed ionic liquids and are referred to as such or as “geminal ionic liquids:” See Armstrong, D. W. et al., Structure and properties of high stability geminal dicationic ionic liquids, J. Amer. Chem. Soc. 2005; 127(2):593-604; and Rogers, R. D. et al., Mercury(II) partitioning from aqueous solutions with a new, hydrophobic ethylene-glycol functionalized bis-imidazolium ionic liquid, Green Chem. 2003; 5:129-135 included herein by reference in its entirety.

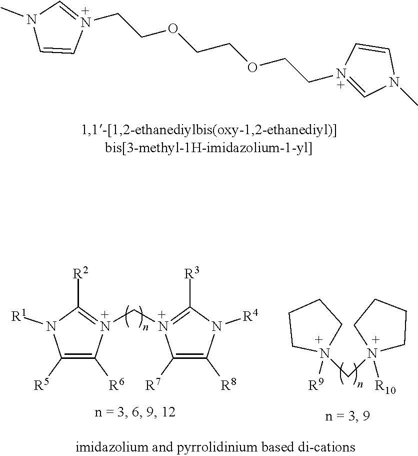

where R¹, R⁴, R⁹, and R¹⁰ comprise a substituted or unsubstituted linear, branched, or cyclic C₁-C₆ alkyl, or substituted or unsubstituted linear, branched, or cyclic C₁-C₆ alkoxy; each R⁵, R⁶, R⁷, and R⁸ is, independently, hydrogen, substituted or unsubstituted linear, branched, or cyclic C₁-C₆ alkyl, substituted or unsubstituted linear, branched, or cyclic C₁-C₆ alkoxy, or substituted or unsubstituted linear or branched, C₁-C₆ alkoxyalkyl.

The choice of the anion in the ionic liquid can be particularly relevant to the rate and level of biopolymer dissolution. While not wishing to be bound by theory, the primary mechanism of solvation of carbohydrates by an ionic liquid is the anion's ability to break the extensive hydrogen-bonding networks by specific interactions with hydroxyl groups. Thus, it is believed that the dissolution of chitin for example is enhanced by increasing the hydrogen bond acceptance and basicity of the anion. By using anions that can accept hydrogen bonds and that are relatively basic, one can not only dissolve pure chitin, but one can dissolve practical grade chitin and even extract chitin from raw chitinous biomass, as described herein. Accordingly, in some examples, the anions are substituted or unsubstituted acyl units R¹⁰CO₂, for example, formate HCO₂⁻, acetate CH₃CO₂⁻ (also noted herein as [OAc]), proprionate, CH₃CH₂CO₂⁻, butyrate CH₃CH₂CH₂CO₂⁻, and benzylate, C₆H₅CO₂⁻; substituted or unsubstituted sulfates: (R¹⁰O)S(═O)₂O⁻; substituted or unsubstituted sulfonates R¹⁰SO₃⁻, for example (CF₃)SO₃⁻; substituted or unsubstituted phosphates: (R¹⁰O)₂P(═O)O⁻; and substituted or unsubstituted carboxylates: (R¹⁰O)C(═O)O⁻. Non-limiting examples of R¹⁰ include hydrogen; substituted or unsubstituted linear branched, and cyclic alkyl; substituted or unsubstituted linear, branched, and cyclic alkoxy; substituted or unsubstituted aryl; substituted or unsubstituted aryloxy; substituted or unsubstituted heterocyclic; substituted or unsubstituted heteroaryl; acyl; silyl; boryl; phosphino; amino; thio; and seleno. In some examples, the anion is C₁₋₆ carboxylate.

Still further examples of anions are deprotonated amino acids, for example, isoleucine, alanine, leucine, asparagine, lysine, aspartic acid, methionine, cysteine, phenylalanine, glutamic acid, threonine, glutamine, tryptophan, glycine, valine, proline, selenocysteine, serine, tyrosine, arginine, histidine, ornithine, and taurine.

It is also contemplated that other anions can be used in some instances, such as halides, (i.e., F⁻, Cl⁻, Br⁻, and I⁻), CO₃²⁻; NO₂⁻, NO₃⁻, SO₄²⁻, CN⁻, arsenate(V), AsX₆ such as AsF₆⁻, and the like; stibate(V) (antimony), SbX₆ such as SbF₆⁻, and the like.

Other non-limiting examples of ionic liquid anions include substituted azolates, that is, five membered heterocyclic aromatic rings that have nitrogen atoms in either positions 1 and 3 (imidazolates); 1, 2, and 3 (1,2,3-triazolates); or 1, 2, 4 (1, 2, 4-triazolate). Substitutions to the ring occur at positions that are not located in nitrogen positions (these are carbon positions) and include CN (cyano-), NO₂ (nitro-), and NH₂ (amino) group appended to the heterocyclic azolate core.

In some examples of suitable ionic liquids, an anion is chosen from formate, acetate, propionate, butyrate, (CF₃)SO₃⁻, (R¹⁰O)S(═O)₂O⁻; (R¹⁰O)₂P(═O)O⁻; (R¹⁰O)C(═O)O⁻; and R¹⁰CO₂⁻; each R¹⁰ is independently C₁-C₆ alkyl. The anion portion of the ionic liquid can be written without the charge, for example, OAc, CHO₂, Cl, Br, RCH₃OPO₂, and PF₆.

In some examples, the ionic liquid comprises a cation selected from the group consisting of:

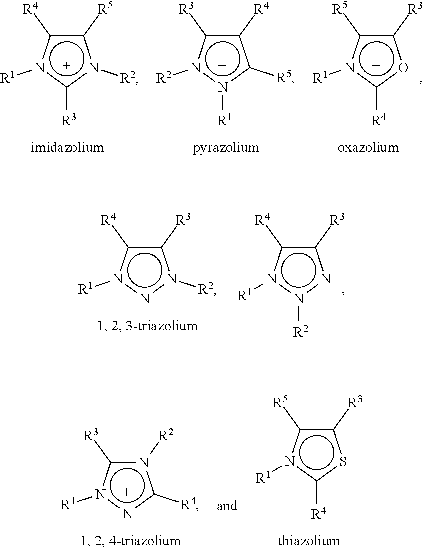

where each R¹ and R² is, independently, a substituted or unsubstituted linear, branched, or cyclic C₁-C₆ alkyl, or substituted or unsubstituted linear, branched, or cyclic C₁-C₆ alkoxy; each R³, R⁴, R⁵, R⁶, R⁷, R⁸, and R⁹ is, independently, hydrogen, substituted or unsubstituted linear, branched, or cyclic C₁-C₆ alkyl, substituted or unsubstituted linear, branched, or cyclic C₁-C₆ alkoxy, or substituted or unsubstituted linear or branched, C₁-C₆ alkoxyalkyl; and

and an anion selected from the group consisting of C₁₋₆ carboxylate, halide, CO₃²; NO₂⁻, NO₃⁻, SO₄²⁻, CN⁻, R¹⁰CO₂⁻, (R¹⁰O)₂P(═O)O⁻, (R¹⁰O)S(═O)₂O⁻, or (R¹⁰O)C(═O)O⁻; where R¹⁰ is hydrogen; substituted or unsubstituted linear, branched, or cyclic alkyl; substituted or unsubstituted linear, branched, or cyclic alkoxy; substituted or unsubstituted aryl; substituted or unsubstituted aryloxy; substituted or unsubstituted heterocyclic; and substituted or unsubstituted heteroaryl.

In preferred examples, the ionic liquid includes a dialkyl-imidazolium carboxylate such as 3-ethyl-1-methyl-imidazolium acetate, [C₂mim]OAc.

Any ionic liquid that effectively dissolves the biopolymer (e.g., cellulose, hemicelluloses, chitin, chitosan, silk, or other natural polysaccharide or polymer) present in the biomass or source of biopolymer can be used in the methods disclosed herein. What is meant by “effectively dissolves” is 25% by weight or more of the biopolymer present is solubilized (e.g., 45% or more, 60% or more, 75% or more, or 90% or more). The formulator can select the ionic liquid for use in the disclosed methods by the one or more factors, for example, solubility of the biomass and/or the biopolymer. The formulator can select the IL for use in this step of the disclosed process by the one or more factors, for example, solubility of the biomass and/or the biopolymer.

3D Printing Compositions

The compositions disclosed herein can be used for three-dimensional (3D) printing. The printing compositions include a biopolymer and optionally a synthetic polymer dissolved in an ionic liquid solvent. In some embodiments, the polymer in the printing compositions disclosed herein can include substantially or completely a biopolymer derived from a biomass. The term “substantially” corresponds to greater than 90 wt %, greater than 95 wt %, or greater than 99 wt %, based on the total weight of polymers in the composition.

In some aspects, the disclosed compositions comprise the biopolymer in an amount of 0.1 wt % or greater (e.g., 0.2 wt % or greater, 0.5 wt % or greater, 0.75 wt % or greater, 1 wt % or greater, 1.5 wt % or greater, 2 wt % or greater, 2.5 wt % or greater, 3 wt % or greater, 3.5 wt % or greater, 4 wt % or greater, 4.5 wt % or greater, 5 wt % or greater, 5.5 wt % or greater, 6 wt % or greater, 7 wt % or greater, 8 wt % or greater, 9 wt % or greater, 10 wt % or greater, or 15 wt % or greater), based on the total weight of the printing composition. In some aspects, the disclosed compositions comprise the biopolymer in an amount of 50 wt % or less (e.g., 45 wt % or less, 40 wt % or less, 35 wt % or less, 30 wt % or less, 25 wt % or less, 20 wt % or less, 15 wt % or less, 10 wt % or less, 7.5 wt % or less, 5 wt % or less, 4.5 wt % or less, 4 wt % or less, 3.5 wt % or less, 3 wt % or less, or 2.5 wt % or less), based on the total weight of the printing composition. In some aspects, the disclosed compositions comprise the biopolymer in an amount of from 0.1 wt % to 50 wt %, from 0.1 wt % to 35 wt %, from 0.1 wt % to 25 wt %, from 0.5 wt % to 20 wt %, from 1 wt % to 20 wt %, from 1 wt % to 50 wt %, from 0.5 wt % to 10 wt %, from 1 wt % to 10 wt %, or from 1 wt % to 5 wt %), based on the total weight of the printing composition. In some aspects, the disclosed compositions comprise the biopolymer in an amount up to about 50% by weight of the composition, up to about 35% by weight of the composition, up to about 25% by weight of the composition, up to about 10% by weight of the composition, or up to about 5% by weight of the composition.

The concentration of the biopolymer and the viscosity of the printing compositions are operational parameters and depend on the method of making and using the printed article. The flow properties and the ability of the printing composition to maintain its shape after extrusion can be tuned by one skilled in the art for example, by variations in concentration of the polymeric components and/or temperature of the printing composition.

Alternately, the printing compositions disclosed herein can include a blend of a synthetic polymer and a biopolymer. For example, the printing compositions disclosed herein can include a blend of one or more synthetic polymers and one or more biopolymers. The weight ratio of synthetic polymer and biopolymer can be 0.1:1 or greater. For example, the weight ratio of biopolymer and synthetic polymer can be 0.5:1 or greater, 1:1 or greater, 1.5:1 or greater, 2:1 or greater, 3:1 or greater, 4:1 or greater, 5:1 or greater, 6:1 or greater, 7:1 or greater, 8:1 or greater, 9:1 or greater, 10:1 or greater, 12:1 or greater, 15:1 or greater, 20:1 or greater, or 25:1 or greater. In certain embodiments, the weight ratio of synthetic polymer and biopolymer can be 30:1 or less, for example, 25:1 or less, 20:1 or less, 18:1 or less, 17:1 or less, 15:1 or less, 12:1 or less, 10:1 or less, 9:1 or less, 8:1 or less, 7:1 or less, 6:1 or less, 5:1 or less, 4:1 or less, 3:1 or less, 2.5:1 or less, 2:1 or less, 1.5:1 or less, or 1:1 or less. In certain embodiments, the weight ratio of synthetic polymer and biopolymer can be from 0.1:1 to 25:1, from 0.1:1 to 20:1, from 1:1 to 25:1, from 1:1 to 20:1, from 1:1 to 18:1, from 1:1 to 15:1, from 2:1 to 10:1, or from 5:1 to 10:1.

In some aspects, the disclosed compositions comprise a blend of the biopolymer and synthetic polymer in an amount of 0.5 wt % or greater (e.g., 0.75 wt % or greater, 1 wt % or greater, 1.5 wt % or greater, 2 wt % or greater, 2.5 wt % or greater, 3 wt % or greater, 3.5 wt % or greater, 4 wt % or greater, 4.5 wt % or greater, 5 wt % or greater, 5.5 wt % or greater, 6 wt % or greater, 7 wt % or greater, 8 wt % or greater, 9 wt % or greater, 10 wt % or greater, 15 wt % or greater, 20 wt % or greater, 25 wt % or greater, 30 wt % or greater, 35 wt % or greater, or 40 wt % or greater), based on the total weight of the printing composition. In some aspects, the disclosed compositions comprise a blend of the biopolymer and synthetic polymer in an amount of 75 wt % or less (e.g., 60 wt % or less, 65 wt % or less, 60 wt % or less, 55 wt % or less, 50 wt % or less, 45 wt % or less, 40 wt % or less, 30 wt % or less, 25 wt % or less, 20 wt % or less, 15 wt % or less, 10 wt % or less, 7.5 wt % or less, 5 wt % or less, 4.5 wt % or less, 4 wt % or less, 3.5 wt % or less, 3 wt % or less, or 2.5 wt % or less), based on the total weight of the printing composition. In some aspects, the disclosed compositions comprise a blend of the biopolymer and synthetic polymer in an amount of from 0.5 wt % to 75 wt %, from 0.5 wt % to 50 wt %, from 0.5 wt % to 25 wt %, from 0.5 wt % to 20 wt %, from 0.5 wt % to 10 wt %, from 1 wt % to 10 wt %, or from 1 wt % to 5 wt %), based on the total weight of the printing composition.

Ionic liquid solvents are used in the printing compositions to solubilize the biopolymer (as well as any other polymer(s) that may be added to the compositions). The ionic liquid solvent can be used alone, or a mixture of two or more solvents may be employed. The term “solvent,” as used herein, may refer to both single-component solvents and solvent mixtures. The solvent may be used in an amount of from 1 to 100 times by weight relative to the total solids content of the printing composition. Typically, the polymer-to-solvent ratio is at least about 0.2, at least about 0.4, at least about 0.6, at least about 0.8, or at least about 1 and may be as high as about 4, as high as about 6, or as high as about 9. In certain embodiments, the polymer-to-solvent ratio is from about 0.2 to about 50, from about 0.2 to about 20, or from about 0.2 to about 4.

The compositions disclosed herein can include (in addition to the biopolymer, the optional synthetic polymer, and the ionic liquid solvent), one or more additives to enhance the flow properties and/or to improve the properties of the printed structure. Exemplary additives can include biologically active compounds, plasticizers, pigments, fire retardants, catalysts, cross-linkers, heat or light stabilizers, organic or inorganic fillers, fiber reinforcements, nanoparticles, additional polymer(s), surfactants, stabilizers, sensitizers, dyes, colorants, ultraviolet radiation absorbers, or combinations thereof.

Organic or inorganic filler particles may be added to the compositions in any amount that does not interfere with printing of the composition. The incorporation of filler particles may improve the structural or aesthetic properties of the printed structure. Suitable filler particles can include carbon nanotubes, graphene nanoplatelets and flakes, graphite powder, clay, metals and nanoparticles agents or dopants, metal oxides, or a combination thereof. The filler particles may have an average particle size in the range from about 1 nm to about 10 μm, and is more typically in the range from about 5 nm to about 500 nm. Thus, the filler particles may be referred to as nano-fillers or nanoparticles in some cases. The filler particles can be present in the compositions in an amount of 1 wt % or greater, such as from 1 wt % to 80 wt %, from 1 wt % to 60 wt %, or from 5 wt % to 50 wt %. In some examples, the printing compositions do not include filler particles.

A dye can be added to the printing composition to increase the absorbance of the compositions. The dye may absorb in the visible part of the spectrum and produce a colored material.

Suitable surfactants that may be employed in the printing composition include, for example, nonionic surfactants, anionic surfactants, cationic surfactants, or combinations thereof. Such nonionic surfactants can include polyoxyethylene alkyl ethers such as polyoxyethylene lauryl ether, polyoxyethylene stearyl ether and polyoxyethylene oleyl ether, polyoxyethylene alkylphenyl ethers such as polyoxyethylene octylphenyl ether and polyoxyethylene nonylphenyl ethers. Further, suitable nonionic ester surfactants may include polyethylene glycol dialkyi esters, such as polyethylene glycol dilaurate and polyethylene glycol distearate. Organosiloxane surfactants may also be suitable for decreasing the surface tension of the printing composition. Additionally, acrylic acid- or methacrylic acid-type polymers and copolymers may serve as suitable surfactants. Suitable amounts of the surfactant may range from about 0.005 to about 1 part by weight per 100 parts by weight of the composition. In addition, antioxidants or defoaming agents can be included as desired to attenuate the composition.

In some examples, a UV photoinitiator may also be employed in the compositions to effect curing. For example, the printing composition can include styrene and divinylbenzene (monomers), Irgacure 819 (UV photoinitiator), isopropylthioxanthone, benzophenone, 2,2-azobisisobutyronitrile, diaryliodonium salts, triarylsulfonium salts, or combinations thereof.

In some aspects of the compositions disclosed herein, the compositions can be formulated as a 3D printing composition consisting essentially of (a) a biopolymer in an amount of from 0.1 wt % to 50 wt %, preferably from 0.1 wt % to 25 wt %, based on the weight of the printing composition, (b) optionally a synthetic polymer, wherein the biopolymer and the synthetic polymer are in a weight ratio of from 1:0.1 to 1:20, from 1:1 to 20:1, or from 1:1 to 10:1, (c) an ionic liquid solvent for dissolving the biopolymer and synthetic polymer, and (d) an additive selected from biologically active compounds, plasticizers, pigments, fire retardants, catalysts, cross-linkers, heat or light stabilizers, organic or inorganic fillers such as nano-fillers, fiber reinforcements, and combinations thereof

Methods

The compositions comprising a biopolymer and an ionic liquid disclosed herein can be used as solvent-based printing compositions that can be 3D printed. The compositions can be printed at room temperature or higher. The 3D printing of the compositions can be used to fabricate various medical devices, such as for use in optoelectronics, photonics, therapeutics, tissue engineering such as intelligent implants, or synthetic biology.

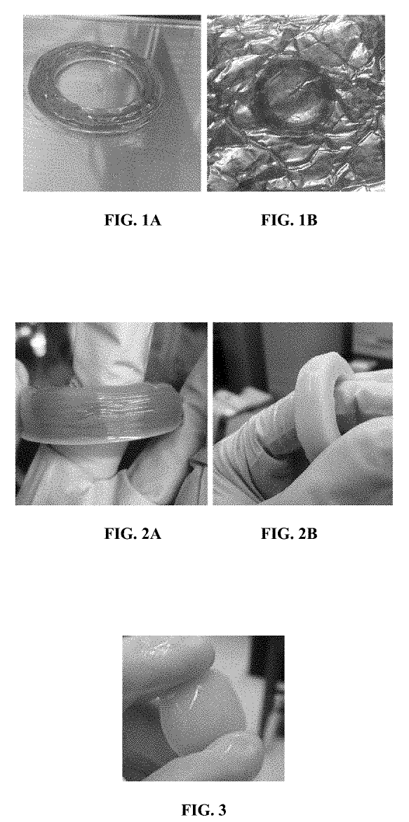

The printing compositions can be readily extruded through a deposition nozzle to form one or more layers (also referred to herein as a filament or continuous filament) that maintains its shape once deposited. According, in some embodiments of the methods described herein, the method does not include depositing the composition into a mold. As shown in FIG. 1, the deposited layers (or filaments) can have a sufficient stiffness to retain its shape and a height. During printing, the deposition nozzle can be moved at a constant or variable print speed while the substrate remains stationary. Alternatively, the substrate may be moved while the deposition nozzle remains stationary, or both the deposition nozzle and the substrate may be moved. One or more layers can be deposited on the substrate in a predetermined 2D or 3D pattern. Using this approach, 3D structures having a wide range of geometries may be built up layer by layer. The substrate is typically a solid, but 3D printing may alternatively be carried out using a gel or viscous liquid as a substrate.

Immediately after printing, the deposited layer may be soft and tacky, although stiff enough to hold its shape. The stiffness of the as-printed layers is high enough that unsupported regions of the printed structure can be formed. For example, rings with 1.5 cm height and diameter of 40 and 20 cm is shown in FIG. 1A. The outer skin of the printed structure quickly hardens, although full drying of the structure can take hours or days, depending on sample thickness and drying conditions.

Prior to extruding the compositions disclosed herein, the biopolymer and the synthetic polymer (when present) can be solubilized in the ionic liquid. In some aspects of the disclosed processes, the biopolymer (for example chitin) can be obtained by directly dissolving or dispersing a pure or practical grade biopolymer or a regenerated biopolymer in an ionic liquid. In other aspects of the disclosed processes, the biopolymer (for example chitin) can be obtained by directly dissolving or dispersing a biopolymer biomass in an ionic liquid. Chitin, for example, obtained by this process is not broken down into small polysaccharide chains as is the case with practical grade or pure grade chitin. As such, direct dissolution of chitin from a biomass allows the formulator to obtain high molecular weight chitin than can be subsequently used to form the printing compositions having different properties than in the case wherein the source of chitin is not directly extracted from a chitinous biomass. The formulator can similarly obtain biopolymers with higher molecular weights, near their original value before extraction, than would otherwise be obtainable. In addition, as disclosed herein, the biomass derived biopolymer can be admixed with one or more adjunct ingredients to form polymeric compositions have properties not obtainable from pure or practical grade chitin.

In some examples, solubilizing the biomass or source of biopolymer and the synthetic polymer (when present) with the ionic liquid can further comprise heating the biomass or source of biopolymer in the ionic liquid to form a mixture. For example, the biomass or source of biopolymer and the synthetic polymer (when present) can be contacted with the ionic liquid at a temperature from about 0° C. to 160° C. In some examples, the methods can further comprise heating the biomass or source of biopolymer and the synthetic polymer (when present) in the ionic liquid at a temperature of about 20° C. or more (e.g., about 25° C. or more, about 30° C. or more, about 35° C. or more, about 40° C. or more, about 45° C. or more, about 50° C. or more, about 55° C. or more, about 60° C. or more, about 65° C. or more, about 70° C. or more, about 75° C. or more, about 80° C. or more, about 85° C. or more, about 90° C. or more, about 95° C. or more, about 100° C. or more, about 105° C. or more, about 110° C. or more, about 115° C. or more, about 120° C. or more, about 125° C. or more, about 130° C. or more, about 135° C. or more, about 140° C. or more, about 145° C. or more, or about 150° C. or more). In some examples, the methods can further comprise heating the biomass or source of biopolymer and the synthetic polymer (when present) in the ionic liquid at a temperature of about 160° C. or less (e.g., about 155° C. or less, about 150° C. or less, about 145° C. or less, about 140° C. or less, about 135° C. or less, about 130° C. or less, about 125° C. or less, about 120° C. or less, about 115° C. or less, about 110° C. or less, about 105° C. or less, about 100° C. or less, about 95° C. or less, about 90° C. or less, about 85° C. or less, about 80° C. or less, about 75° C. or less, about 70° C. or less, about 65° C. or less, about 60° C. or less, about 55° C. or less, about 50° C. or less, about 45° C. or less, about 40° C. or less, about 35° C. or less, about 30° C. or less, or about 25° C. or less).

The temperature at which the biomass or source of biopolymer and the synthetic polymer (when present) in the ionic liquid is heated can range from any of the minimum values described above to any of the maximum values described above. For example, the methods can further comprise heating the biomass or source of biopolymer and the synthetic polymer (when present) in the ionic liquid at a temperature from about 20° C. to about 150° C. (e.g., from about 20° C. to about 100° C., from about 20° C. to about 60° C., from about 60° C. to about 100° C., from about 20° C. to about 40° C., from about 40° C. to about 60° C., from about 60° C. to about 80° C., from about 80° C. to about 100° C., or from about 30° C. to about 90° C.).

In some examples, microwave heating can be used to extract and/or dissolve the biomass or source of biopolymer and the synthetic polymer (when present). In one example, the biomass or source of biopolymer and the synthetic polymer (when present) can be combined with an ionic liquid or an ionic liquid/co-solvent. In other examples, the biomass or source of biopolymer and the synthetic polymer (when present) does not include a co-solvent. The co-solvent can include an organic co-solvent. The term “organic co-solvent” as used herein refers to a component of the compositions which is present in excess and which physical state is in the same as that of the ionic liquid. The organic co-solvent may be capable of at least partially dissolving the biomass, source of biopolymer, and/or the synthetic polymer. The organic co-solvent expressly excludes ionic liquids as described herein. In certain embodiments, the co-solvent can include 1-butanol, dimethyl sulfoxide (DMSO), or combinations thereof. The mixture can be charged to a source of microwave radiation and the mixture heated to extract and/or dissolve the biopolymer. In one example, short 1 to 5 second pulses are used, however, and pulse time can be used to extract and/or dissolve the biopolymer and the synthetic polymer (when present), i.e., 1 second, 2 seconds, 3 seconds, 4 seconds, or 5 seconds, or any fractional part thereof. For these examples, the temperature can be critical; however, microwave heating provides an efficient and desirable method for extracting and/or dissolving high molecular weight biopolymers like chitin from a biomass or source of chitin.

The microwave irradiation can be conducted for a total irradiation time of 1 minute or more (e.g., 2 minutes or more, 3 minutes or more, 4 minutes or more, 5 minutes or more, 6 minutes or more, 7 minutes or more, 8 minutes or more, 9 minutes or more, 10 minutes or more, 15 minutes or more, 20 minutes or more, or 25 minutes or more). In some examples, the microwave irradiation can be conducted for a total irradiation time of 30 minutes or less (e.g., 25 minutes or less, 20 minutes or less, 15 minutes or less, 10 minutes or less, 9 minutes or less, 8 minutes or less, 7 minutes or less, 6 minutes or less, 5 minutes or less, 4 minutes or less, 3 minutes or less, or 2 minutes or less). The total irradiation time of the microwave irradiation can range from any of the minimum values described above to any of the maximum values described above. For example, the microwave irradiation can be conducted for a total irradiation time of from 1 minute to 30 minutes (e.g., from 1 minute to 15 minutes, from 15 minutes to 30 minutes, from 1 minute to 25 minutes, from 1 minute to 20 minutes, from 1 minute to 10 minutes, from 1 minute to 5 minutes, or from 3 minutes to 5 minutes).

In some examples, the microwave irradiation is conducted with 1-30 second pulses for a total of 1-60 min irradiation time with stirring between the pulses. In some examples, the microwave irradiation is conducted with 2-3 second pulses for a total of 3-5 min irradiation time with stirring between the pulses.

The methods can further comprise agitating the mixture of biopolymer and ionic liquid. Agitating the mixture can be accomplished by any means known in the art. In some examples, agitating the mixture can comprise stirring the mixture.

In some examples, when the biopolymer is chitin, the source of chitin is pure chitin, for example, pure chitin obtained from crab shells, C9752, available from Sigma, St. Louis, Mo. In other examples, the source of chitin can be practical grade chitin obtained from crab shells, C7170, available from Sigma, St. Louis, Mo. In further examples, the source of chitin can be chitinous biomass, such as shrimp shells that are removed from the meat by peeling and processed to insure all shrimp meat is removed. However, any biomass comprising chitin or mixtures of chitin and chitosan, or mixtures of chitin, chitosan, and other polysaccharides can be used as the source of chitin.

As described herein, the printing compositions can comprise from about 0.1 wt % to about 75 wt % of biopolymer (e.g., about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 30 wt %, about 35 wt %, about 40 wt %, about 45 wt %, or about 50 wt %, where any of the stated values can form an upper or lower endpoint of a range).

Methods of making a printed article from the compositions described herein are also described herein. The method can be based on solvent casting or extrusion-based 3D printing. The method can include extruding the printing composition as described herein from a deposition nozzle moving relative to a substrate. One or more layers comprising the composition can be deposited in a predetermined pattern on the substrate to form a 3D printed body. The composition can be treated to form the 3D article.

The extrusion can be carried out at ambient temperature or greater. For example, the extrusion can be carried out from ambient temperature (about 18° C. to about 25° C.) to 150° C., from 20° C. to 100° C., from 20° C. to 60° C., or from 30° C. to 50° C. The deposition nozzle can be moved and the layers can be deposited at print speeds as high as about 3 m/s, although more typical print speeds range from 10 μm/s to 500 mm/s, from 100 μm/s to 100 mm/s, from 1 mm/s to 100 mm/s, or from 10 mm/s to 50 mm/s. The deposition nozzle can be microscale in size with an inner diameter or width ranging from 0.5 μm to 2,000 μm (2 mm), preferably from 10 μm to 1,000 μm or from 10 μm to 500 μm. In certain embodiments, larger nozzle diameter can be used. For example, the deposition nozzle can be scaled in size with an inner diameter or width up to 10 cm. Depending on the nozzle size as well as the injection pressure and nozzle translation speed, the extruded layer can have a width or diameter ranging from about 1 μm to about 2 mm.

The printing composition fed to the deposition nozzle can be housed in a syringe barrel connected to the nozzle by a suitable connector. The extrusion of each of the layer can take place under an applied pressure of from 1 psi (6.89 kPa) to 500 psi (3,447 kPa) or from 10 psi (68.95 kPa) to 200 psi (1,379 kPa). The pressure during extrusion may be constant or it may be varied. By using alternative pressure sources, pressures of higher than 500 psi (3,447 kPa) and/or less than 1 psi (6.89 kPa) can be applied during printing. A variable pressure can yield a continuous filament having a diameter that varies along the length of the filament. During the extrusion and deposition of each layer, the nozzle can be moved along a predetermined path with a positional accuracy of within ±100 μm. Accordingly, the layers can be deposited with a positional accuracy of within ±200 μm, preferably within ±100 μm, more preferably within ±10 μm.

The printed layers deposited on the substrate can be understood to encompass a single continuous layer of a desired length or multiple extruded layers having end-to-end contact once deposited to form a continuous layer of the desired dimensions. A layer of any length can be produced by halting deposition after the desired length of the layer has been reached. The desired length can depend on the print path and/or the geometry of the structure being fabricated. A layer of any width can be produced by varying the diameter of the nozzle. The desired width can depend on the print path and/or the geometry of the structure being fabricated.

The treatment of the composition after extrusion can include allowing the composition to solidify. In some examples, the composition can be allowed to solidify at 35° C. or less. In some examples, the composition can be allowed to solidify at ambient temperature or less, such as less than 25° C. or less than 20° C. In some examples, the composition can be allowed to solidify above ambient temperature, such as greater than 25° C., greater than 30° C., or greater than 35° C.

The treatment of the composition can include coagulating the biopolymer and/or removing the ionic liquid solvent. In some examples, the biopolymer can be coagulated using a non-solvent. The term “non-solvent” as used herein refers to a solvent or mixture of solvents in which the provided biopolymer and synthetic polymer (when present) are insoluble or poorly soluble. A suitable non-solvent can be defined by showing a maximum solubility of 5 g/L, preferably a maximum solubility of 3 g/L. In certain embodiments, the non-solvent can include an aqueous mixture. In some examples, the non-solvent can be water. The non-solvent can further include solutions of kosmotropic salt. As used herein, a “kosmotropic salt” is any salt that contributes to the stability and structure of water-water interactions, e.g., that causes water molecules to favorably interact. In some examples, the kosmotropic salt comprises an anion and a cation, wherein the anion is selected from the group consisting of CO₃²⁻, SO₄²⁻, PO₄³⁻, HPO₄²⁻, H₂PO₄⁻, Cl⁻, HCO₃⁻, F⁻, OH⁻, and S₂O₃²⁻. In some examples, the kosmotropic salt comprises K₃PO₄, K₂HPO₄, Na₃PO₄, Na₂HPO₄, NaH₂PO₄, K₂CO₃, KOH, NaOH, KHCO₃, NaHCO₃, Na₂S₂O₃, or a combination thereof. The concentration of the kosmotropic salt in the non-solvent can, for example, be from 0 wt % to 60 wt % or from 5 wt % to 50 wt %. The methods described herein can include contacting the printed composition with an aqueous non-solvent to coagulate the biopolymer. In some examples, the printed composition is contacted with the aqueous solution by submerging the printed composition in the non-solvent.

The methods can further comprise removing the ionic liquid from the printed composition. FIGS. 2A-2B show printed material after 30 min in a coagulation bath and after complete removal of the ionic liquid. Methods for removing the ionic liquid from the printed composition can include washing the printed composition with the non-solvent. For example, the printed composition can be submerged in the non-solvent for at least 30 mins, at least 1 hr, at least 1.5 hrs, at least 2 hrs, at least 6 hrs, at least 8 hrs, at least 10 hrs, at least 12 hrs, at least 15 hrs, at least 20 hrs, or at least 24 hrs to coagulate the biopolymer and remove the ionic liquid solvent. The non-solvent can be removed from the printed composition by drying (e.g., freeze drying) and/or heating (e.g., evaporation) to remove the non-solvent.

The treatment to coagulate the biopolymer and remove the ionic liquid solvent typically occurs after extruding the composition through the deposition nozzle and depositing the one or more layers on the substrate. It is also contemplated, however, that the treatment may occur after extrusion but prior to or during deposition of the one or more layers.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures and other reaction ranges and conditions that can be used to optimize the product purity and yield obtained from the described process. Only reasonable and routine experimentation will be required to optimize such process conditions.

### Example 1: Method to Manufacture Biopolymeric Materials and Biopolymer Composites Through 3D-Printing from Ionic Liquids

### Summary of Example

This example is directed to a 3D-printing process of biopolymers. The process provides 3D structures from either pure biopolymer(s) or their blends with synthetic polymers. The process also allows inclusion of additives or inorganic fillers (e.g., biologically active compounds, graphene, nanoparticles agents or dopants, inorganics, plasticizers, pigments, fire retardants, heat and light stabilizers, fillers and fiber reinforcements, etc.). Specifically, the present example dissolves biopolymers in ionic liquids for fabricating 3D-printed structures. The technology is based on solution processing of biopolymers using ionic liquids (ILs), through biopolymer dissolution, followed by layer-by-layer deposition, solidification (once deposited onto surface), and cooling. After the 3D shape is created and solidified, the print is coagulated in an aqueous bath, followed by multiple washing steps to remove the ionic liquid. The materials after coagulation maintain/preserve the initial shape formed in layer-by-layer print fashion. The quality (thickness) of the print is controlled by the extruder nozzle diameter and printing speed. The exemplified 3D-printing technique (biopolymers printing from IL) provides a universal method for printing materials from biopolymers or their blends that cannot be melt-processed or printed using common organic solvents.

Currently, biopolymers manufactured into advanced materials through the use of harsh chemicals to degrade the biopolymers are unsuitable for manufacturing biomedical materials. Therefore, alternative approaches to process biopolymers into advanced materials suitable for medical applications are needed.

3D-printing is an emerging technology that uses computer-created 3D models to build solid materials in a ‘layer build up’ fashion, for on-demand production of final products or parts. The technology has found broad application in healthcare, automotive, aerospace and bioprinting industries. There are multiple 3D-printing techniques such as ink-jet printing, fused deposition and laser sintering that are suitable for processing different types of materials available on the market. However, the most common technology suitable for polymer printing is fused deposition, where the polymer is heated above its melting point, melted and then solidified, once printed. Normally, the polymers used for this type of printing are acrylonitrile-butadiene-styrene (ABS) and polystyrene and nylon, which are petroleum-based and have slow degradation rate in the environment. Additionally, fused deposition technology is suitable for a number of polymers due to the need of melt-processing (i.e., those that melt at relatively low, process-affordable, temperature). Biopolymers including chitin or cellulose derived directly from biomass generally cannot be printed using fused deposition due to their decomposition prior to melting.

In recent years, paste extrusion has been shown to be suitable for printing paste or gel-like materials such as ceramics, silicone and cellulose or chitin derivatives. Using this extrusion method, it becomes possible to 3D-print chitin and cellulose nanocrystals, and their derivatives from volatile organic solvents (VOCs) or aqueous slurries. The solidification of the print using VOCs or water was done through rapid solvent evaporation or post-print freeze-drying step. While printing of nanocrystals or their VOC soluble derivatives into 3D structure was shown to be feasible, printing of biomass-extracted biopolymers in their native form using the same approach remains a challenge due to their insolubility in aqueous solutions and VOCs.

Exemplified is a 3D-printing methodology using ionic liquids platform for biopolymers that cannot be melt-processed or solubilized by other aqueous or organic solvents. In this example, the method allows 3D-printing of high molecular weight biopolymers without need of their chemical modification directly onto solid support using paste extrusion technique. The present technology provides the use of certain biopolymers (e.g., chitin and cellulose) for preparing printable IL-based solutions. In particular, the present example encompasses the recognition that biopolymers can be dissolved in ILs and then used to make 3D-prints. The unique material features of biopolymer-based solutions allow incorporation of more than one biopolymers, combination of the biopolymer with synthetic polymer(s), as well as the use of a variety of additives (e.g., nanoparticles agents or dopants, graphene, inorganics), which are stabilized by the IL. The 3D printed materials are useful for a wide range of applications, including but not limited to, optoelectonics, photonics, therapeutics, tissue engineering such as intelligent implants, synthetic biology, and a variety of consumer products.

Biopolymer Dissolution:

Solutions of biopolymers in 1-ethyl-3-methylimidazolium acetate ([C₂mim][OAc]) were prepared. For this, chitin was first extracted from biomass waste (shrimp shells) according to a previously reported procedure (Shamshina, J. L. et al., J. Mater. Chem. B 2014, 2, 3924-3936). Specifically, regenerated chitin powder was thermally dissolved by stirring in an oil bath at 100° C. for 10-24 h to yield solutions with chitin concentration of 2.5-3 wt %.

Preparation of Biopolymer Composites:

Also prepared were composite chitin-poly(lactic acid) (PLA) solutions. The solutions were prepared by simultaneous thermal dissolution of regenerated chitin powder and PLA at different chitin to PLA ratio (from 1:1 to 1:9), at 100° C. over 15 h using oil bath. While the ratio between PLA to chitin was kept constant, the polymer mass load was varied. Specifically, the mass of PLA added in the IL was from 1.77 wt % to 27 wt % and the mass of regenerated chitin was from 1.77 wt % to 3 wt %.

3D Printing of Biopolymers and Biopolymers Composites:

The 3D-printing of biopolymers and biopolymer composites solubilized in IL was carried using a Printrbot Simple Metal 3D printer equipped with heated paste extruder (available from Printrbot company) in which the rubber plunger cap in the syringe was substituted with custom-made Teflon analog.

The prepared solutions were transferred into a 60 mL plastic syringe right after the dissolution step and placed in the preheated extruder (35-50° C.). The print shape was defined by a 3D model developed using Fusion 360 Software and print parameters were controlled by Cura 1.5. The print layer thickness was controlled by using different size of blunt plastic needles (14G-22G). The accuracy of the print was controlled by the printing speed (10-50 mm/s). The temperature of the extruder was varied from 35-50° C. to achieve sufficient solution flow. Depending on the biopolymer concentration in the IL, printed layers were solidified either at room temperature or below. 3D printing of chitin: regenerated chitin solution was prepared with a chitin load of 3 wt % in [C₂mim][OAc]. Rings with 1.5 cm height and diameter of 40 and 20 cm were used as 3D models. For the printing, the extruder was kept at 40° C. and the print speed rate was 30 mm/s. The prints were immersed in an aqueous solvent for coagulating the biopolymer. The print on a solid support (glass) after immersion into a coagulation bath is shown on FIG. 1B. The chitin ring with smaller diameter (20 cm) and 1.5 cm height was printed at 35° C. The printing resulted in a stable layer adhesion during print and after coagulation in water. FIGS. 21-2B show the material after 30 min in coagulation bath and after complete removal of IL.

A cubical model as shown in FIG. 3 was also printed from the same chitin solution.

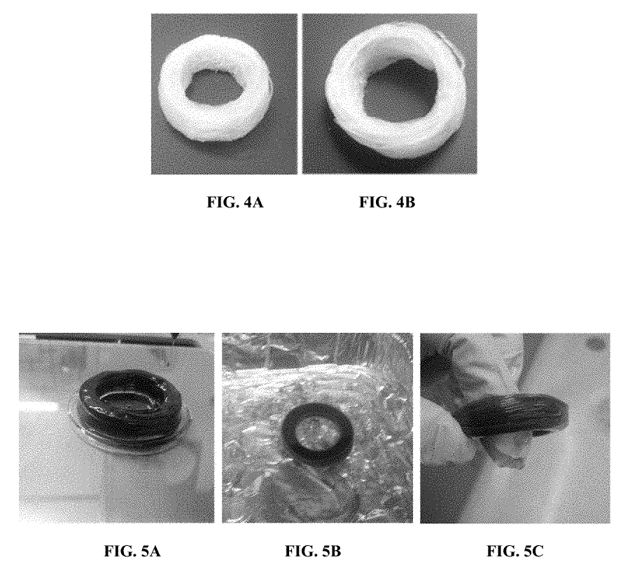

The printed materials were freeze-dried from aqueous solution. The 3D printed chitin material after freeze-drying is shown in FIGS. 4A-4B.

3D Printing Composites:

3D-prints from chitin-PLA composites were also prepared at 1 to 1 biopolymer ratios with each polymer mass load of 3 wt % (total weight % of loaded polymers was 6 wt %). As a 3D-printing model, the ring with 1.5 cm height and 20 cm diameter was used to test the quality of layer adhesion. The print was processed in a layer-by-layer fashion and the shape was maintained after the print, while keeping at room temperature (FIG. 5A). The print was coagulated in an aqueous bath and after ˜10 min the layers are still seen on the print surface and the print remained intact (FIG. 5B). IL was removed from the material by washing the print with deionized waster (DI), FIG. 5C.

### Example 2

Materials:

Deionized (DI) water was obtained from a commercial deionizer (Culligan, Northbrook, Ill., USA) with specific resistivity of 16.82 MΩ·cm at 25° C. The ionic liquid, 1-ethyl-3-methylimidazolium acetate ([C₂mim][OAc], purity >95%) was purchased from IoLiTec, Inc. (Tuscaloosa, Ala., USA). Poly(L-lactic acid) (PLA) with molecular weight of ˜700,000 (6.5 dl/g), was purchased from Polysciences, Inc. (Warrington, Pa.).

Solutions of Biomass:

Solution of shrimp shell extract was prepared accordingly to previously published procedure. Briefly, shrimp shells (2 wt %) in [C₂mim][OAc] were prepared by heating using microwave irradiation with 2 sec pulses with manual stirring for 6 min. For the first 30 sec, the heating time was in 10 sec pulses. After the desired microwave time was reached, the solution was transferred into centrifuge tubes and centrifuged at 3000 rpm for 20 min to remove undissolved residues. Centrifuged solutions were poured into tubes (decanted from a residue remained after centrifugation) and were used for obtaining regenerated chitin.

Regenerated Chitin:

The solution of processed biomass (decanted from the residues) as obtained above (60 g) was coagulated in 1 L of deionized water (DI) during constant stirring and left overnight to remove IL from coagulated chitin. The chitin obtained was transferred into centrifuge tubes to remove any remaining aqueous phase. The fresh DI water was added followed by sonication and centrifugation at 3000 rpm for 15 min. The steps were repeated 10 times. Regenerated chitin was oven dried at 60° C. Regenerated chitin was dried and sieved to obtained chitin particles size <250 μm using the same procedure described above.

Chitin and Chitin PLA Composite Solutions for 3D-Printing:

The regenerated chitin was thermally dissolved in [C₂mim][OAc] using an oil-bath at 100° C. with stirring and heating overnight to yield solutions with the desired concentrations (from 2.5 to 3 wt %). For 3D-printing of chitin-PLA composites, chitin and PLA powder were simultaneously dissolved in [C₂mim][OAc] under constant stirring at 100° C. for 15 h. The prepared solutions had the following mass ratio: 9:1 and 1:1 between PLA and chitin. While the ratio between PLA to chitin was kept constant, polymer mass load was varied. Specifically, mass of PLA added into IL was from 27 wt % to 1.77 wt %; mass of regenerated chitin was from 3 wt % to 1.77 wt %.

3D-Printing:

3D-prints were processed with Printrbot Simple Metal 3D printer equipped with heated paste extruder (Printrbot, Lincoln, Calif.) equipped with plastic syringe (60 mL, Soft-Ject Luer Lock syringe). Disposable plastic blunt needles (14G to 22G) were purchased from Amazon. Prior to printing, the rubber plunger in the syringe was substituted with custom made Teflon analog. The syringe was loaded with solutions for printing, when solutions were hot (˜80° C., to reduce solution viscosity) followed by syringe insertion into pre-heated extruder (35-50° C.).

The models for 3D-printing were designed with Fusion 360 Software and the designed file was converted into stl format for the printer. The 3D printer operation was controlled using Cura 1.5 Software (Ultimaker) that allows controlling temperature and accuracy of the print through adjusting print speed (10-50 mm/s).

Coagulation and Drying of Printed Materials:

After the 3D-printing, the prints were coagulated in an aqueous bath filled with deionized water (DI). The print was washed with DI water 10 times and soaking overnight in DI water. After the IL was removed, the print was freeze-dried using Labconco Freezone freeze dryer system (Labconco, Kansas City, Mo.).

What is claimed is:

1. A method of printing a three-dimensional (3D) article comprising:
extruding a printing composition from a deposition nozzle moving relative to a substrate, the printing composition comprising a biopolymer dissolved in an ionic liquid solvent;
depositing one or more layers comprising the printing composition in a predetermined pattern on the substrate; and
treating the one or more layers to form the 3D article.

2. The method of claim 1, wherein the biopolymer is selected from starch, pectin, chitin, chitosan, alginate, silk, elastin, collagen, gelatin, hemicellulose, lignin, cellulose, lignocellulose, or combinations thereof.

3. The method of claim 1, wherein the biopolymer is present in the printing composition in an amount from 0.1 wt % to 50 wt %.

4. The method of claim 1, wherein the printing composition further comprises a synthetic polymer.

5. The method of claim 4, wherein the synthetic polymer includes a polylactic acid, a polyester, a polyacrylonitrile, a poly(N,N-dimethyl acrylamide), a poly(1-vinylpyrrolidinone), a polyhydroxyethylmethacrylate, a polymethylmethacrylate, a poly(vinylidene fluoride), a polycaprolactone, a polyalkylene glycol, a polyurethane, or a combination thereof.

6. The method of claim 4, wherein the biopolymer and the synthetic polymer are in a weight ratio of from 1:0.1 to 1:20.

7. The method of claim 1, wherein the ionic liquid comprises:
a cation selected from the group consisting of: 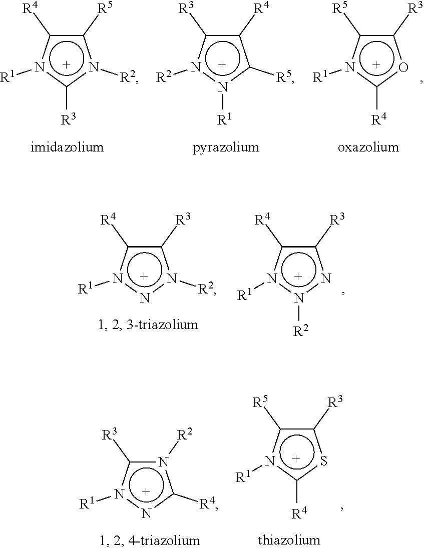
wherein each R¹ and R² is, independently, a substituted or unsubstituted linear, branched, or cyclic C₁-C₆alkyl, or substituted or unsubstituted linear, branched, or cyclic C₁-C₆ alkoxy;
each R³, R⁴, and R⁵ is, independently, hydrogen, substituted or unsubstituted linear, branched, or cyclic C₁-C₆ alkyl, substituted or unsubstituted linear, branched, or cyclic C₁-C₆ alkoxy, or substituted or unsubstituted linear or branched, C₁-C₆ alkoxyalkyl; and
an anion selected from the group consisting of C₁₋₆ carboxylate, halide, CO₃²⁻ NO₂⁻, NO₃⁻, SO₄²⁻, CN⁻, R¹⁰CO₂⁻, (R¹⁰O)₂P(═O)O⁻, (R¹⁰O)S(═O)₂O—, or (R¹⁰O)C(═O)O⁻; where R¹⁰ is hydrogen; substituted or unsubstituted linear, branched, or cyclic alkyl; substituted or unsubstituted linear, branched, or cyclic alkoxy; substituted or unsubstituted aryl; substituted or unsubstituted aryloxy; substituted or unsubstituted heterocyclic; and substituted or unsubstituted heteroaryl.

8. The method of claim 1, wherein the printing composition further comprises a biologically active compound, a plasticizer, a pigment, a fire retardant, a catalyst, a cross-linker, a heat or light stabilizer, an organic or inorganic filler such as a nano-filler, a fiber reinforcement, or a combination thereof.

9. The method of claim 1, wherein extruding is carried out from 20° C. to 150° C.

10. The method of claim 1, wherein the deposition nozzle moves relative to the substrate at a print speed from about 10 mm/s to about 50 mm/s.

11. The method of claim 1, wherein the method further comprises allowing the one or more layers to solidify at from 0° C. to 35° C. prior to treating the one or more layers.

12. The method of claim 1, wherein treating the one or more layers comprises coagulating the biopolymer and/or removing the ionic liquid solvent.

13. The method of claim 12, wherein coagulating the biopolymer and/or removing the ionic liquid solvent includes contacting the one or more layers with a non-solvent.

14. The method of claim 13, wherein the non-solvent is an aqueous solvent.

15. A 3D printed article derived from a method of claim 1, wherein the article is for use in optoelectronics, photonics, therapeutics, tissue engineering such as intelligent implants, or synthetic biology.

16. A printing composition consisting essentially of:
a biopolymer present in an amount of from 0.1 wt % to 50 wt %, based on the weight of the printing composition;
a synthetic polymer, wherein the biopolymer and the synthetic polymer are in a weight ratio of from 1:0.1 to 1:20;
an ionic liquid solvent; and
a 3D printing additive.

17. The printing composition of claim 16, wherein the biopolymer includes starch, pectin, chitin, chitosan, alginate, silk, elastin, collagen, gelatin, hemicellulose, lignin, cellulose, lignocellulose, or combinations thereof.

18. The printing composition of claim 16, wherein the synthetic polymer includes a polylactic acid, a polyester, a polyacrylonitrile, a poly(N,N-dimethyl acrylamide), a poly(1-vinylpyrrolidinone), a polyhydroxyethylmethacrylate, a polymethylmethacrylate, a poly(vinylidene fluoride), a polycaprolactone, a polyalkylene glycol, a polyurethane, or a combination thereof.

19. The printing composition of claim 16, wherein the synthetic polymer is present an amount of from 1 wt % to 50 wt %, based on the weight of the printing composition.

20. The printing composition of claim 16, wherein the ionic liquid comprises a cation selected from the group consisting of: 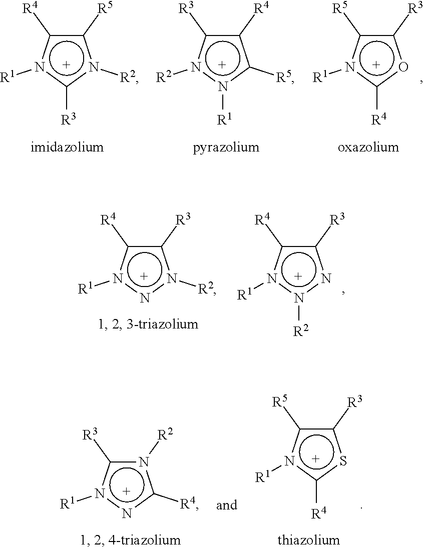
where each R¹ and R² is, independently, a substituted or unsubstituted linear, branched, or cyclic C₁-C₆ alkyl, or substituted or unsubstituted linear, branched, or cyclic C₁-C₆ alkoxy;
each R³, R⁴, and R⁵ is, independently, hydrogen, substituted or unsubstituted linear, branched, or cyclic C₁-C₆ alkyl, substituted or unsubstituted linear, branched, or cyclic C₁-C₆ alkoxy, or substituted or unsubstituted linear or branched, C₁-C₆ alkoxyalkyl; and
an anion selected from the group consisting of C₁₋₆ carboxylate, halide, CO₃²⁻ NO₂⁻, NO₃⁻, SO₄²⁻, CN⁻, R¹⁰CO₂⁻, (R¹⁰O)₂P(═O)O⁻, (R¹⁰O)S(═O)₂O⁻, or (R¹⁰O)C(═O)O⁻; where R¹⁰ is hydrogen; substituted or unsubstituted linear, branched, or cyclic alkyl; substituted or unsubstituted linear, branched, or cyclic alkoxy; substituted or unsubstituted aryl; substituted or unsubstituted aryloxy; substituted or unsubstituted heterocyclic; and substituted or unsubstituted heteroaryl.

21. The printing composition of claim 16, wherein the ionic liquid includes a 1-alkyl-3-alkyl imidazolium C₁-C₆ carboxylate.

22. The printing composition of claim 16, wherein the printing composition does not include an organic co-solvent.
